(19) United States
(12) Patent Application Publication
(10) Pub. No.: US 2024/0024008 A1
(43) Pub. Date: Jan. 25, 2024

(54) FRACTURE DISTRACTOR PLIERS

(71) Applicants: Brent Lane Norris, Jenks, OK (US); Ryan Stafford, Fort Wayne, IN (US); Baylor Norris, Jenks, OK (US); Lane Norris, Jenks, OK (US)

(72) Inventors: Brent Lane Norris, Jenks, OK (US); Ryan Stafford, Fort Wayne, IN (US); Baylor Norris, Jenks, OK (US); Lane Norris, Jenks, OK (US)

(21) Appl. No.: 18/223,629

(22) Filed: Jul. 19, 2023

Related U.S. Application Data
(60) Provisional application No. 63/390,358, filed on Jul. 19, 2022.

(51) Int. Cl. A61B 17/88; A61B 17/16

(52) U.S. Cl. CPC A61B 17/885; A61B 17/1697; A61B 2017/00407

(57) ABSTRACT

Fracture distractor pliers comprising: a first arm with a first end and an opposing second end, where the first end of the first arm terminates in a slot capable of engaging a screw head; and a second arm with a first end and an opposing second end, where the first end of the second arm terminates in a recess capable of engaging a plate. The first arm and the second arm may be pivotally joined. The fracture distractor pliers may facilitate precise distraction of fracture fragments of the larger long bones, assist provisional fracture stabilization to facilitate final fixation, and ergonomically overcome compressive forces at a fracture site.

### CROSS REFERENCE

This application is based on and claims priority to U.S. Provisional Patent Application No. 63/390,358 filed Jul. 19, 2022.

### FIELD OF THE INVENTION

This invention relates generally to surgical tools, and more particularly, but not by way of limitation, to fracture distractor pliers.

### DESCRIPTION OF THE RELATED ART

Unstable bone fractures sometimes must be reduced and fixed with a plate during surgery. The patient's muscles may cause the fracture to shorten, necessitating the surgeon to lengthen the fracture manually. This is generally done using clamps, but clamps often do not hold the bone well, leading to surgeon fatigue.

Based on the foregoing, it is desirable to provide a device that gradually tensions a fracture.

It is further desirable for the device to facilitate precise distraction of fracture fragments of the larger long bones.

It is further desirable for the device to assist provisional fracture stabilization to facilitate final fixation.

It is further desirable for the device to ergonomically overcome compressive forces at a fracture site.

### SUMMARY OF THE INVENTION

In general, in a first aspect, the invention relates to fracture distractor pliers comprising: a first arm with a first end and an opposing second end, where the first end of the first arm terminates in a slot capable of engaging a screw head; and a second arm with a first end and an opposing second end, where the first end of the second arm terminates in a recess capable of engaging a plate. The first arm and the second arm may be pivotally joined.

The groove and the recess may each project perpendicularly from their respective arm and in opposite direction to each other. The first arm and the second arm may be pivotally joined by a hinge. The fracture distractor pliers may further comprise a ratchet mechanism, which may be graduated. The fracture distractor pliers may further comprise a locking mechanism, such as a thumb screw, capable of locking the arms into place relative to each other such that they cannot pivot. The second end of the first arm and the second end of the second arm may form handles, such that squeezing the handles together separates the first end of the first arm from the first end of the second arm. The fracture distractor pliers may be made of surgical stainless steel and may be capable of being sterilized and re-used. The first arm may have a hole capable of receiving a K-wire.

In a second aspect, the invention relates to a method of fracture distraction, the method comprising: attaching a plate on a proximal side of a fracture; placing a screw on a distal side of the fracture; engaging the screw with a groove at a first end of a first arm of fracture distractor pliers; engaging the plate with a recess at a first end of a second arm of the fracture distractor pliers, where the first arm and the second arm are pivotally joined; squeezing a second end of the first arm toward a second end of the second arm such that the groove and the recess, and consequently the screw and plate, separate; when the screw and plate, and consequently the distal side of the fracture and the proximal side of the fracture to which they are respectively attached, are sufficiently separated, attaching the plate to the distal side of the fracture; and disengaging the fracture distractor pliers from the screw and the plate and removing the fracture distractor pliers.

The method may further comprise engaging a locking mechanism on the fracture distractor pliers when the screw and plate are sufficiently separated and before attaching the plate, and releasing the locking mechanism after attaching the plate to the distal side of the fracture and before disengaging the fracture distractor pliers from the screw and plate. The may further comprise allowing a ratcheting mechanism to engage while squeezing the second end of the first arm toward the second end of the second arm. The method may further comprise sterilizing and re-using the fracture distractor pliers. The method may further comprise attaching a K-wire on the distal side of the fracture and sliding the K-wire into a hole in the first arm of the fracture distractor pliers to form an additional point of fixation.

### BRIEF DESCRIPTION OF THE DRAWINGS

FIG. 1 is a perspective view of the fracture distractor pliers;

FIG. 2 is a perspective view of the fracture distractor pliers, showing the slot;

FIG. 3 is a perspective view of the fracture distractor pliers, showing the recess;

FIG. 4 is a front view of the fracture distractor pliers;

FIG. 5 is a bottom view of the fracture distractor pliers;

FIG. 6 is a proximal side view of the fracture distractor pliers;

FIG. 7 is a distal side view of the fracture distractor pliers;

FIG. 8 is a top view of the fracture distractor pliers;

FIG. 9 is a bottom view of the fracture distractor pliers;

FIG. 10 is a perspective view of an alternate version of the fracture distractor pliers;

FIG. 11 is a close-up perspective view of one side of the first end of the fracture distractor pliers; and

FIG. 12 is a close-up perspective view of the opposite side of the first end of the fracture distractor pliers.

Other advantages and features will be apparent from the following description and from the claims.

### DETAILED DESCRIPTION OF THE INVENTION

The devices and methods discussed herein are merely illustrative of specific manners in which to make and use this invention and are not to be interpreted as limiting in scope.

While the devices and methods have been described with a certain degree of particularity, it is to be noted that many modifications may be made in the details of the construction and the arrangement of the devices and components without departing from the spirit and scope of this disclosure. It is understood that the devices and methods are not limited to the embodiments set forth herein for purposes of exemplification.

In general, in a first aspect, the invention relates to fracture distractor pliers 1, as shown in the Figures. The fracture distractor pliers 1 may facilitate precise distraction of fracture fragments of the larger long bones. The purpose of the fracture distractor pliers 1 is to assist provisional fracture stabilization to facilitate final fixation, and to ergonomically overcome compressive forces at a fracture site. The fracture distractor pliers 1 may be most useful for the proximal humerus and for the distal tibia.

The fracture distractor pliers 1 may have two arms 2. One arm 2 may terminate at a first end in a groove 4 capable of capturing a screw head to leverage for distraction, while the other arm 2 may terminate at its first end in a recess 6 capable of capturing one end of a plate to leverage for distraction. Both the groove 4 and the recess 6 may project perpendicularly from the arms 2, and in opposite directions to each other. The arms 2 may be pivotally joined by a hinge 8. The opposing ends of the arms 2 may not terminate where they join, but rather may extend further, forming handles 9 that may be squeezed to separate the first ends.

The screw head may vary in size from 2.7 mm to 4.5 mm. The plate may likewise vary from 2.7 mm to 4.5 mm. Alternately, the fracture distractor pliers 1 may have a different sized groove 4 and a different sized recess 6 to be suitable for use with any desired size of screw head and plate.

The arm 2 that terminates at a first end in a groove 4 capable of capturing a screw head may further have a hole 11 capable of receiving a K-wire. The hole 11 may be oblong.

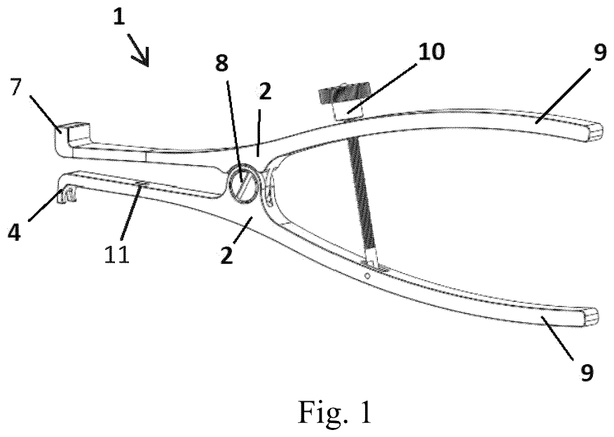

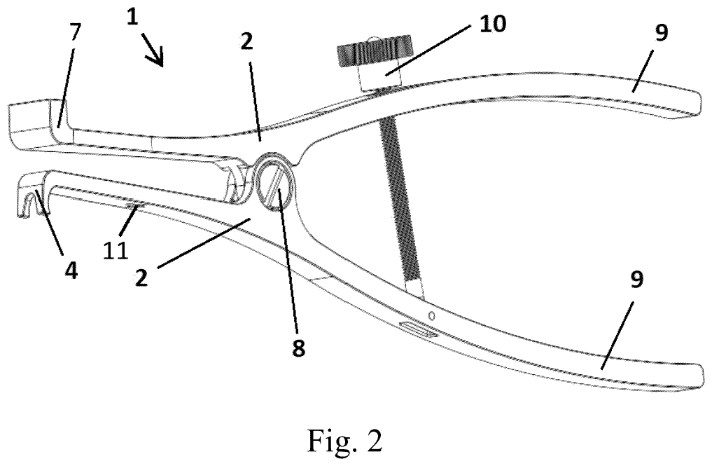

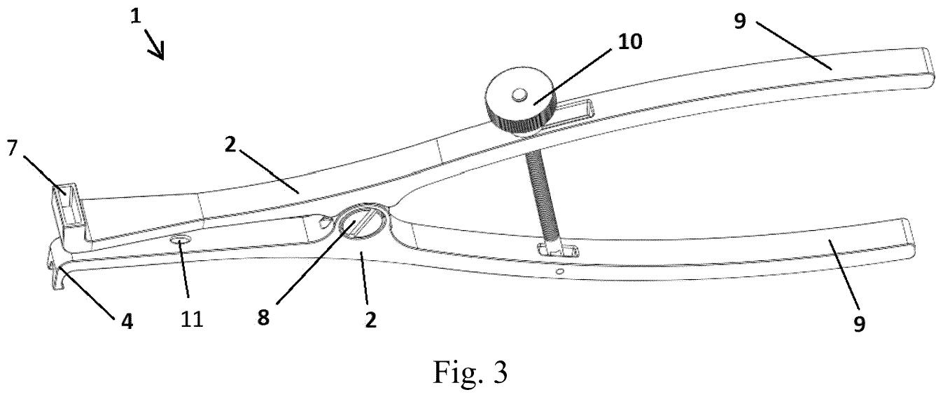

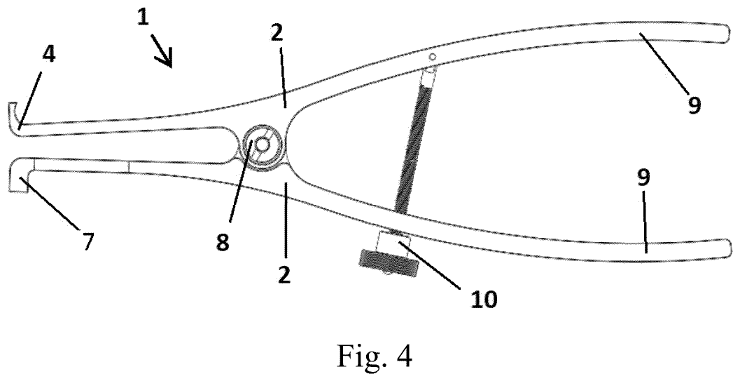

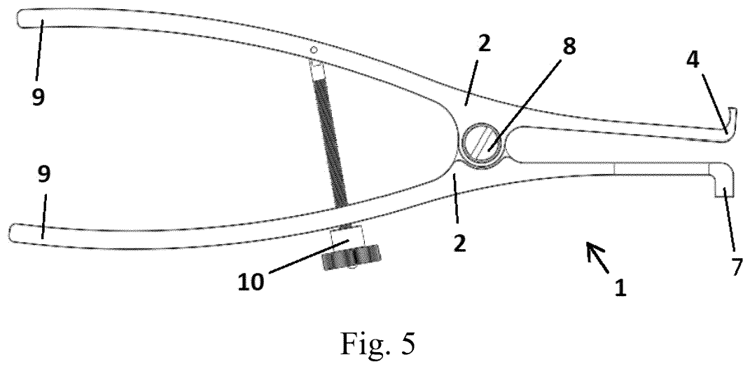

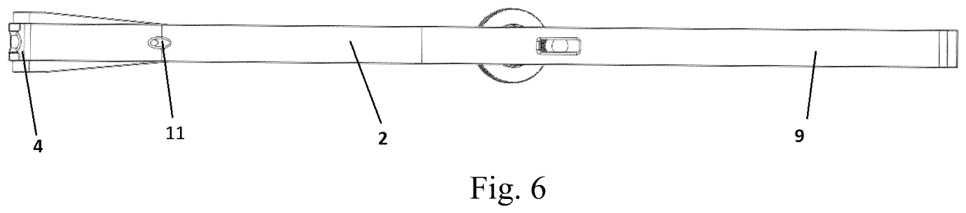

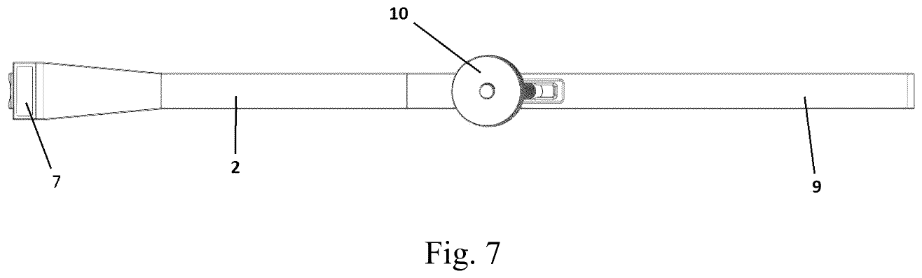

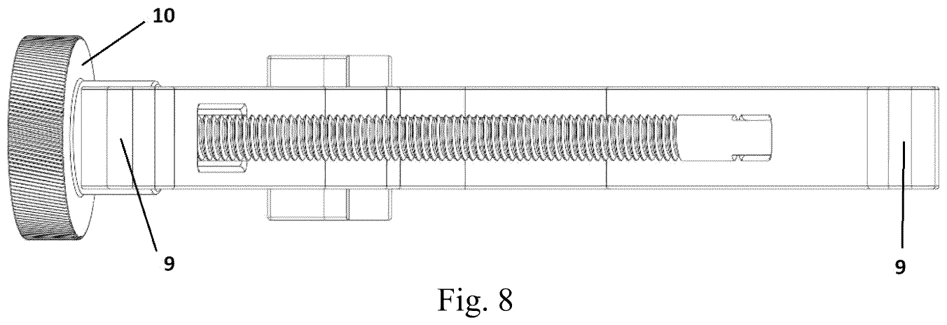

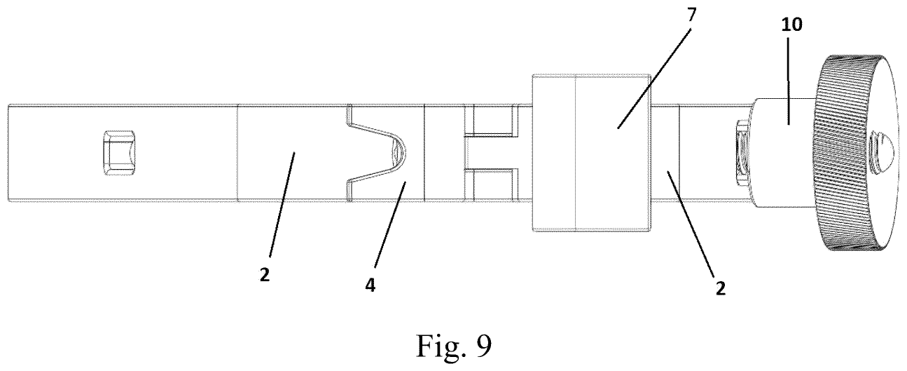

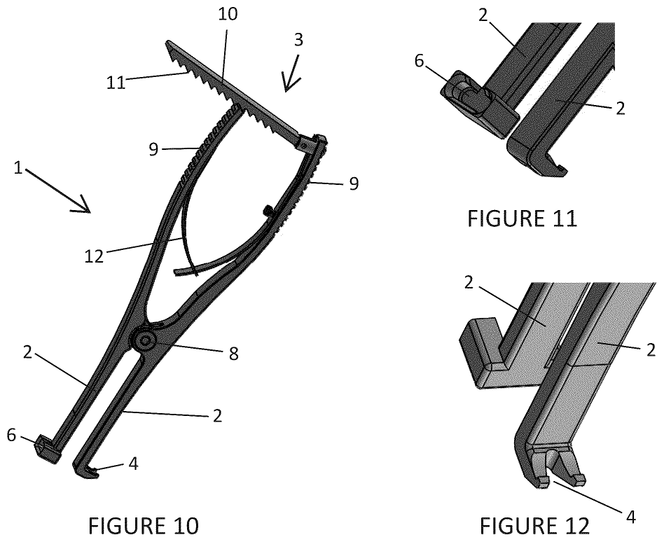

The fracture distractor pliers 1 may further comprise a locking mechanism 10 to lock the fracture distractor pliers 1 into place with the arms 2 at a desired distance. For example, as show in FIGS. 1 through 9, the locking mechanism 10 may be a thumb screw locking mechanism. Alternately, as shown in FIG. 10, the locking mechanism 10 may be part of a ratchet mechanism 3. The ratchet mechanism 3 may be graduated. The locking mechanism 10 may be a flip-down bar that holds the two handles 9 at a set distance, where the flip-down bar has multiple angled teeth 11 to allow the handles 9, and thus the arms 2, to be held at various desired distances from each other. The ratchet mechanism 3 may further comprise a pair of spring arms 12 tending to push the handles 9, and thus the arms 2, away from each other when not locked by the locking mechanism 10.

The fracture distractor pliers 1 may be made of surgical stainless steel, or any other desired material. The device may be capable of being sterilized and re-used.

During use, a surgeon may attach a plate on the proximal side of a fracture and may place a screw on the distal side of the fracture. The surgeon may engage the screw with the groove 4 and the plate with the recess 6, and may then squeeze the handles 9 to pivot the arms 2 and force the groove 4 and recess 6, and consequently the screw and plate and the bone sections to which they are attached, apart. This gradually tensions the fracture. As the arms 2 pivot, the ratcheting mechanism 3 may engage, preventing the fracture distractor pliers 1 from releasing. When the fracture fragments are sufficiently separated, the surgeon may secure the locking mechanism 10 and proceed to secure the plate on the distal side of the fracture. Upon completion, the surgeon may release the locking mechanism 10, release the ratcheting mechanism 3, and disengage the fracture distractor pliers 1 from the screw and plate. After use, the fracture distractor pliers 1 may be sterilized and re-used.

If the fracture distractor pliers 1 have a hole 11, the surgeon may attach a K-wire to the bone on the distal side of the fracture and may slide the K-wire into the hole 11 at an angle for an additional point of fixation to stabilize and further define the plane.

The fracture distractor pliers 1 may ratchet and lock similarly to a lamina spreader. A lamina spreader, however, is not useful for fracture distraction, as it does not engage with the screw or the plate well. The groove 4 and recess 6 of the fracture distractor pliers 1, on the other hand, are specifically designed to engage with the screw and the plate.

While the fracture distractor pliers 1 are described herein with reference to distraction of fractures, the pliers 1 may be equally useful for other applications, both surgical and non-surgical.

Whereas, the devices and methods have been described in relation to the drawings and claims, it should be understood that other and further modifications, apart from those shown or suggested herein, may be made within the spirit and scope of this invention.

What is claimed is:

1. Fracture distractor pliers comprising:
2 a first arm with a first end and an opposing second end, where the first end of the 3 first arm terminates in a slot capable of engaging a screw head; and
4 a second arm with a first end and an opposing second end, where the first end of the second arm terminates in a recess capable of engaging a plate;
6 where the first arm and the second arm are pivotally joined.

2. The fracture distractor pliers of claim 1 where the groove and the recess each project perpendicularly from their respective arm and in opposite direction to each other.

3. The fracture distractor pliers of claim 1 where the first arm and the second arm are pivotally joined by a hinge.

4. The fracture distractor pliers of claim 1 further comprising a ratchet mechanism.

5. The fracture distractor pliers of claim 4 where the ratchet mechanism is graduated.

6. The fracture distractor pliers of claim 1 further comprising a locking mechanism capable of locking the arms into place relative to each other such that they cannot pivot.

7. The fracture distractor pliers of claim 6 where the locking mechanism is a thumb screw.

8. The fracture distractor pliers of claim 1 where the second end of the first arm and the second end of the second arm form handles, such that squeezing the handles together separates the first end of the first arm from the first end of the second arm.

9. The fracture distractor pliers of claim 1 where the fracture distractor pliers are made of surgical stainless steel.

10. The fracture distractor pliers of claim 1 where the fracture distractor pliers are capable of being sterilized and re-used.

11. The fracture distractor pliers of claim 1 where the first arm has a hole capable of receiving a K-wire.

12. A method of fracture distraction, the method comprising:
attaching a plate on a proximal side of a fracture;
placing a screw on a distal side of the fracture;
engaging the screw with a groove at a first end of a first arm of fracture distractor pliers;
engaging the plate with a recess at a first end of a second arm of the fracture distractor pliers, where the first arm and the second arm are pivotally joined;
squeezing a second end of the first arm toward a second end of the second arm such that the groove and the recess, and consequently the screw and plate, separate;
when the screw and plate, and consequently the distal side of the fracture and the proximal side of the fracture to which they are respectively attached, are sufficiently separated, attaching the plate to the distal side of the fracture; and
disengaging the fracture distractor pliers from the screw and the plate and removing the fracture distractor pliers.

13. The method of claim 12 further comprising engaging a locking mechanism on the fracture distractor pliers when the screw and plate are sufficiently separated and before attaching the plate, and releasing the locking mechanism after attaching the plate to the distal side of the fracture and before disengaging the fracture distractor pliers from the screw and plate.

14. The method of claim 12 further comprising allowing a ratcheting mechanism to engage while squeezing the second end of the first arm toward the second end of the second arm.

15. The method of claim 12 further comprising sterilizing and re-using the fracture distractor pliers.

16. The method of claim 12 further comprising:
attaching a K-wire on the distal side of the fracture; and
sliding the K-wire into a hole in the first arm of the fracture distractor pliers to form an additional point of fixation.
